(19) United States
(12) Patent Application Publication
(10) Pub. No.: US 2017/0340738 A1
(43) Pub. Date: Nov. 30, 2017

(54) STABLE ACIDIC INSULIN FORMULATIONS

(71) Applicant: MERCK SHARP & DOHME CORP., Rahway, NJ (US)

(72) Inventors: Maya S. Salnikova, Doylestown, PA (US); Christina R. Vessely, Golden, CO (US); Jeffrey T. Blue, Telford, PA (US)

(73) Assignee: Merck Sharp & Dohme Corp., Rahway, NJ (US)

(21) Appl. No.: 15/594,966

(22) Filed: May 15, 2017

Related U.S. Application Data
(63) Continuation of application No. 14/509,468, filed on Oct. 8, 2014, now abandoned.
Application No. 14/509,468 is a continuation of application No. 13/790,260, filed on Mar. 8, 2013, now abandoned.
(60) Provisional application No. 61/609,528, filed on Mar. 12, 2012.

(51) Int. Cl. A61K 47/10; A61K 38/28; A61K 9/08

(52) U.S. Cl. CPC A61K 47/10; A61K 9/08; A61K 38/28

(57) ABSTRACT

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

### CROSS REFERENCE TO RELATED APPLICATIONS

This application is a continuation of U.S. Ser. No. 14/509,468, filed Oct. 8, 2014, and which is a continuation of U.S. Ser. No. 13/790,260, filed Mar. 8, 2013, and which claims benefit of U.S. Provisional Application No. 61/609,528 filed Mar. 12, 2012, the content of which is hereby incorporated by reference in its entirety.

### BACKGROUND OF THE INVENTION

### (1) Field of the Invention

The present invention relates to acidic insulin formulations comprising surfactants or excipients that prevent agitation-induced aggregation of the insulin. In particular, the present invention relates to Gly(A21), Arg(B31), Arg(B32)-human insulin (insulin glargine) formulations comprising polyethylene glycol 400 or trehalose/proline.

### (2) Description of Related Art

Insulin is a peptide hormone that is essential for maintaining proper glucose levels in most higher eukaryotes, including humans. Diabetes is a disease in which the individual cannot make insulin or develops insulin resistance. Type I diabetes is a form of diabetes mellitus that results from autoimmune destruction of insulin-producing beta cells of the pancreas and Type II diabetes is a metabolic disorder that is characterized by high blood glucose in the context of insulin resistance and relative insulin deficiency. Left untreated, an individual with Type I or Type II diabetes will die. While not a cure, insulin is effective for lowering glucose in virtually all forms of diabetes.

Gly(A21), Arg(B31), Arg(B32)-human insulin or insulin glargine is marketed worldwide under the trade name LANTUS. It is a long acting insulin that provides 24 hour basal coverage and is administered once daily subcutaneously (See U.S. Pat. No. 5,656,722). Insulin glargine is injected as an acidic, clear solution that precipitates due to its solution properties in the physiological pH range of the subcutaneous tissue as a stable hexamer associate. Insulin glargine is injected once daily and is distinguishable from other long-acting insulins by its flat serum profile and the reduced risk of nightly hypoglycemia associated therewith (Schubert-Zsilavecz et al., 2:125-130(2001)).

Insulin glargine is marketed worldwide in vials for use with a syringe and in cartridges for use in pen-type injectors. The specific preparation of insulin glargine, which leads to the prolonged duration of action, is characterized, in contrast to previously described preparations, by a clear solution having an acidic pH. However, insulins at acidic pH show a decreased stability and an increased propensity toward aggregation upon thermal and physicomechanical stress, which results in turbidity and precipitation (particle formation) of the insulin glargine (Brange et al., J. Ph. Sci 86:517-525(1997)). The propensity toward aggregation can additionally be promoted by hydrophobic surfaces which are in contact with the solution (Sluzky et al., Proc. Natl. Acad. Sci. 88:9377-9381 (1991). Surfaces which can be considered as hydrophobic may be the glass vessels of the preparations, the stopper material of the sealing caps or the boundary surface of the solution with the air (air liquid interface). Moreover, very fine silicone oil droplets can function as additional hydrophobic aggregation nuclei that are found in siliconized insulin syringes for the daily insulin administration.

Currently, insulin glargine marketed in vials has a formulation that differs from the currently marketed cartridge formulation by only the inclusion of the surfactant, polysorbate 20, in the vial formulation. The polysorbate 20 is included in the vial for the purpose of preventing agitation-induced aggregation of the protein (See U.S. Pat. Nos. 7,476,652 and 7,713,930). Other excipients in both formulations include 100 IU/mL (3.64 mg/mL) insulin glargine, 20 mg/mL (85%) glycerol, 2.7 mg/mL m-cresol, and 30 μg/mL zinc. The formulation is supplied unbuffered at pH 4. While insulin glargine appears to be stabilized in formulations comprising polysorbate 20, there remains a need for identifying other formulations that stabilize the insulin glargine. For example, more recent formulations of insulin glargine include insulin glargine at a concentration of about 300 U/mL (U.S. Published Application No. 20110301081), methionine as a stabilizer (International Application No. WO 2011/003822), and sulfobutyl ether derivatives of β-cyclodextrin as a stabilizer (International Patent Application No. WO2011144674).

The present invention was thus based on the object of finding preparations for acid-soluble insulins such as insulin glargine that provide long-term stability.

### BRIEF SUMMARY OF THE INVENTION

The present invention provides formulations that can greatly increase the stability of aqueous acidic insulin preparations and thus preparations can be produced which have greater stability to hydrophobic aggregation nuclei for several months. In general, the present invention provides aqueous acidic insulin formulations comprising either (i) PEG 400 at a concentration of about 20 μg/mL to about 200 μg/mL or (ii) about 30 mg/mL trehalose and about 50 mM proline, wherein the insulin derivative has an isoelectric point between 5 and 8.5, or a physiologically tolerated salt thereof, and is of the Formula

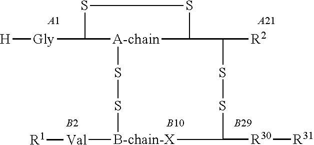

in which R¹ at position B1 denotes H or H-Phe; R² at position A21 denotes a genetically encodable L-amino acid selected from the group consisting of Gly, Ala, Val, Leu, Ile, Pro, Phe, Trp, Met, Ser, Thr, Cys, Tyr, Asp, and Glu; R³⁰ represents the residue of a neutral genetically encodable L-amino acid selected from the group consisting of Ala, Thr, and Ser; R³¹ represents 1, 2, or 3 neutral or basic α-amino acids, wherein at least one of the α-amino acids is selected from the group consisting of Arg, Lys, Hyl, Orn, Cit, and His; X represents His at position B10; and the sequences A1 to A20 and B1 to B29 in the Formula correspond to a mammalian insulin, excluding those insulin derivatives in which simultaneously: R¹ at position B1 denotes Phe; and R³¹ is one α-amino acid having a terminal carboxyl group. Optionally, the amino acid(s) at position A21 and/or the C-terminal amino acid of R³¹ is(are) amidated at the carboxy terminus.

In a specific aspects, the acidic insulin is Gly(A21), Arg(B31), Arg(B32)-human insulin (insulin glargine) or Gly(A21), Arg(B31), Arg(B32)-NH₂-human insulin (B32 amidated insulin glargine). In particular aspects, the Gly(A21), Arg(B31), Arg(B32)-human insulin is present in a concentration of about 100 to 1000 U/mL or of about 100 or 300 U/mL.

In further aspects, the aqueous pharmaceutical formulation has a pH in the acidic range from 1 to 6.8. In further aspects, the formulation comprises at least one preservative, which in particular embodiments may be selected from phenols. In a further aspect, the preservative is m-cresol, chlorocresol, benzyl alcohol, or paraben.

In further aspects, the formulation includes zinc. In further aspects, the formulation includes at least one isotonicitizing agent. In further aspects of formulations containing PEG 400, the one isotonicizing agent may be mannitol, sorbitol, lactose, dextrose, trehalose, sodium chloride, or glycerol. In further aspects of formulations containing Trehalose and proline, the one isotonicizing agent may be mannitol, sorbitol, lactose, dextrose, sodium chloride, or glycerol.

In further aspects, the formulation may further include a buffer, which in particular embodiments may be selected from TRIS, phosphate, citrate, acetate, and glycylglycine. When the buffer is present it may have a concentration of about 5-250 mM

The pharmaceutical formulation as claimed in claim 1, wherein the PEG MW 400 is present in a concentration of about 5-400 or 20-200 μg/mL.

In particular aspects, the PEG MW 400 is present in a concentration of about 20 μg/mL.

In particular aspects of the formulation, the trehalose is present at 30 mg/mL and the proline is present at 100-400 or 50 mM.

The pharmaceutical formulation may further comprise one or more excipients chosen from acids, alkalis and salts. In particular aspects, the excipient is NaCl which is present in a concentration of up to 150 mM.

### BRIEF DESCRIPTION OF THE DRAWINGS

FIG. 1—Typical SEC-HPLC Chromatogram of stressed (heat+agitation) vs. unstressed material are shown by the black and dotted-line traces respectively. The top chromatogram is a full scale image. The bottom chromatogram is a magnification of the baseline around the monomer peak.

FIG. 2A-2B—HP-SEC results following storage at 2-8° C., no agitation. FIG. 2A shows the accumulation of dimmer over time. FIG. 2B shows the percent recovery of soluble protein over time.

FIG. 3A-3B—HP-SEC results following storage at 25° C., no agitation. FIG. 3A shows the accumulation of dimmer over time. FIG. 3B shows the percent recovery of soluble protein over time.

FIG. 4A-4B—HP-SEC results following storage at 40° C., no agitation. FIG. 4A shows the accumulation of dimmer over time. FIG. 4B shows the percent recovery of soluble protein over time.

FIG. 5A-5B—HP-SEC results following agitation at 25° C. or 40° C. FIG. 5A shows the accumulation of dimmer over time. FIG. 5B shows the percent recovery of soluble protein over time.

FIG. 6—Typical RP-HPLC Chromatogram of stressed (heat+agitation) vs. unstressed material are shown by the black and dotted-line traces respectively. The top chromatogram is a full-scale image. The bottom chromatogram is a magnification of the baseline around the monomer peak.

FIG. 7—RP-HPLC results following storage at 2-8° C., no agitation.

FIG. 8—RP-HPLC results following storage at 25° C., no agitation.

FIG. 9—RP-HPLC results following storage at 40° C., no agitation.

FIG. 10—RP-HPLC results following agitation.

FIG. 11—Typical CEX-HPLC Chromatogram. The top chromatogram is a full-scale image. The bottom chromatogram is a magnification of the baseline around the monomer peak.

FIG. 12—CEX-HPLC results following storage at 2-8° C., no agitation.

FIG. 13—CEX-HPLC results following storage at 25° C., no agitation.

FIG. 14—CEX-HPLC results following storage at 40° C., no agitation.

FIG. 15—CEX-HPLC results following agitation.

FIG. 16—Microflow Imaging results following 4 weeks storage at 2-8° C., no agitation.

FIG. 17—Microflow Imaging results following 4 weeks storage at 25° C., no agitation.

FIG. 18—Microflow Imaging results following 4 weeks storage at 40° C., no agitation.

FIG. 19—Microflow Imaging results following agitation at 40° C.

FIG. 20—Image results following agitation at 40° C., Originator Cartridge Formulation (Form 1).

FIG. 21—Image results following agitation at 40° C., NaCl/Arginine (gelled) Formulation (Form 10)

FIG. 22—Image results following agitation at 40° C., High concentration Span Formulation (Form 12).

FIG. 23—In-vitro Precipitation Assay.

FIG. 24—Insulin Tolerance Test, LANTUS formulation comparison.

### DETAILED DESCRIPTION OF THE INVENTION

The present invention provides formulations that can greatly increase the stability of aqueous acidic insulin preparations and thus preparations can be produced which have greater stability to hydrophobic aggregation nuclei for several months. In general, the present invention provides stability to aqueous acidic insulin preparations comprising an insulin derivative having an isoelectric point between 5 and 8.5, or a physiologically tolerated salt thereof, of the Formula

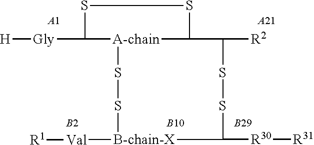

in which R¹ at position B1 denotes H or H-Phe; R² at position A21 denotes a genetically encodable L-amino acid selected from the group consisting of Gly, Ala, Val, Leu, Ile, Pro, Phe, Trp, Met, Ser, Thr, Cys, Tyr, Asp, and Glu; R³⁰ represents the residue of a neutral genetically encodable L-amino acid selected from the group consisting of Ala, Thr, and Ser; R³¹ represents 1, 2, or 3 neutral or basic α-amino acids, wherein at least one of the α-amino acids is selected from the group consisting of Arg, Lys, Hyl, Orn, Cit, and His; X represents His at position B10; and the sequences A1 to A20 and B1 to B29 in the Formula correspond to a mammalian insulin, excluding those insulin derivatives in which simultaneously: R¹ at position B1 denotes Phe; and R³¹ is one α-amino acid having a terminal carboxyl group. Optionally, the amino acid(s) at position A21 and/or the C-terminal amino acid of R³¹ is(are) amidated at the carboxy terminus.

In a specific aspects, the acidic insulin is Gly(A21), Arg(B31), Arg(B32)-human insulin (insulin glargine) or Gly(A21), Arg(B31), Arg(B32)-NH₂-human insulin (B32 amidated insulin glargine).

The inventors have found that aqueous acidic insulin preparations, for example aqueous preparations comprising insulin glargine, can be stabilized against aggregation of the insulin during long-time storage up to at least 18 months by including in the preparation polyethylene glycol molecular weight (MW) 400 (PEG 400) or trehalose and proline. As shown in the examples, aqueous preparations of insulin glargine comprising PEG 400 at a concentration of about 20 μg/mL to about 200 μg/mL was effective at stabilizing the preparation for up to 18 months at 2-8° C. The examples further show that aqueous preparations of insulin glargine comprising about 30 mg/mL trehalose and 50 mM proline was also effective at stabilizing the preparation for up to 18 months at 2-8° C.

The preparations can additionally optionally contain preservatives (e.g. phenol, m-cresol, parabens), isotonicitizing agents (e.g. mannitol, sorbitol, lactose, dextrose, sodium chloride, glycerol), buffer substances, salts, acids and alkalis and also further excipients. These substances can in each case be present individually or alternatively as mixtures. Glycerol, dextrose, lactose, sorbitol and mannitol are customarily present in the pharmaceutical preparation in a concentration of 100-250 mM, NaCl in a concentration of up to 150 mM. Buffer substances, such as, for example, phosphate, acetate, citrate, arginine, glycylglycine or TRIS (i.e. 2-amino-2-hydroxymethyl-1,3-propanediol) buffer and corresponding salts, are present in a concentration of 5-250 mM, preferably 10-100 mM. Further excipients may include salts.

PEG has been used in a number of formulations of human products, including Flebogamma®5% Rx, an IV infusion of IgG. PEG is used in the manufacturing process and may be present in the product as a residual material, with an acceptance criteria of <6 mg/mL. Another product is Gammagard S/D, which is also an IV infusion of IgG. The PDR lists a PEG concentration of about 2 mg/mL. A third example of a product from the PDR is Aralast® RX. This is an alpha 1-proteinase inhibitor delivered by IV infusion. Again, PEG appears to be a residual material from manufacturing process, and is acceptable at concentrations of <112 mg/mL. Patent application GB2107185A (1982) discloses insulin preparations comprising polyether, preferably with a MW between 5,000 and 1000,000. While no examples were found that specifically listed PEG 400 as a formulation excipient, PEG 400 is included on the Food and Drug Administration GRAS list, so it is considered of low risk as an excipient.

### Example 1

This example shows the development of stable acidic insulin formulations.

### Materials and Equipment

Table 1 lists the raw materials and primary packaging components used for the study.

TABLE 1
Materials required for preparation of DP Formulation

| Raw material | Spec #/Part Number/Lot number |
|---|---|
| LANTUS Cartridges, | Lot # 40C243 |
| 3 mL | |
| Glycerol | Fisher G31-1 Lot# 084957 |
| m-cresol | Spectrum Part# C2273, lot # |
| Zinc chloride | Fisher Lot# 086692 |
| Trehalose | Ferro Pfanstiehl, lot# 1060480 |
| Proline | Ajinimoto lot# R028E005 |
| Arginine | Ajinimoto part #L-arg |
| NaCl | JT Baker Lot# H49H04 |
| Polysorbate 20 | Croda Lot# G13583 |
| Span 40 | Sigma part# 388920 Lot# 07315BH |
| Polyethylene glycol | Croda Super refined Lot#P3266 |
| 400 (PEG400) | |
| Vials | Material description: 2-ml vial, 13-mm diameter |
| | Type I, clear glass vial, West part # 68000314 |
| Stoppers | West serum stoppers with flurotec, 13 mm Part # |
| | 19500040. |
| Cap/Seals | 13 mm flip off seals, blue, West part# 54130045 |

Insulin Glargine forms a depot upon injection into the body due to the precipitation of hexameric units of protein. The proper hexamer formation for insulin molecules requires the inclusion of Zn and an aromatic molecule, for example, m-cresol. In this example, Zn and m-cresol were present at concentrations of 30 μg/mL and 2.7 mg/mL respectively, in all formulations included in the preformulation study. Additionally, because insulin glargine has an isoelectric point around pH 7, the formulation is adjusted to about pH 4; therefore, all formulations included in this preformulation study were adjusted to pH 4. The preformulation study investigated a total of 14 formulations in an effort to screen compatibility with a variety of salts and sugars, amino acids, and surfactants. The commercially obtained cartridge and vial formulations were included as controls. The formulations included in the study are listed in Table 2.

TABLE 2
Formulation Parameters

| Formulation # | Tonicity Modifier | Surfactant | Stabilizer |
|---|---|---|---|
| 1 (commercial | 20 mg/mL glycerol | | |
| cartridge) | | | |
| 2 (commercial | 20 mg/mL glycerol | 20 μg/mL | |
| vial) | | Polysorbate 20 | |
| 3 | 20 mg/mL glycerol | 20 μg/mL Span 40 | |
| 4 | 20 mg/mL sorbitol | 20 μg/mL Span 40 | |
| 5 | 30 mg/mL Trehalose | 20 μg/mL Span 40 | |
| 6 | 30 mg/mL Trehalose | | |
| 7 | 10 mg/mL Trehalose + | | |
| | 100 mM NaCl | | |
| 8 | 30 mg/mL Trehalose | | 50 mM |
| | | | Arginine |
| 9 | 30 mg/mL Trehalose | | 50 mM |
| | | | Proline |
| 10 | 100 mM NaCl | | 50 mM |
| | | | Arginine |
| 11 | 100 mM NaCl | | 50 mM |
| | | | Proline |
| 12 | 20 mg/mL glycerol | 200 μg/mL Span 40 | |
| 13 | 20 mg/mL glycerol | 20 μg/mL PEG | |
| 14 | 20 mg/mL glycerol | 200 μg/mL PEG | |

In order initiate preformulation studies prior to the completion of process development for biosimilar insulin glargine and the production of demonstration batches, 15 boxes of cartridges (3 mL/cartridge, 5 cartridges per box, LANTUS Lot #40C243) were obtained from a commercial supplier. Cartridges were chosen over vials due to the exclusion of the surfactant polysorbate 20 from the cartridge configuration. The other excipients in the cartridge formulation were removed by dialysis.

Insulin glargine-containing solution was removed from the cartridges and pooled together to provide a pooled solution volume of approximately 225 mL. The pooled solution was transferred to dialysis cartridges (ThermoScientific/Pierce, part #66230, 2K MWCO, volume 30 mL). This process was performed for the preparation of all formulations, including numbers 1 and 2, so that each would experience similar levels of handling prior to study initiation. The dialysis cartridges were transferred to dialysis buckets containing about 4 L of dialysis solution. Four different dialysis solutions were used, and the pooled solution was divided among them as follows:

Dialysis solution 1: 30 μg/mL Zinc, 20 mg/mL Sorbitol, pH 4. 30 mL of pooled solution was added for dialysis.

Dialysis solution 2: 30 μg/mL Zinc, 20 mg/mL Glycerol, pH 4. 90 mL of pooled solution was added for dialysis.

Dialysis solution 3: 30 μg/mL Zinc, 100 mM NaCl, pH 4. 30 mL of pooled solution was added for dialysis.

Dialysis solution 4: 30 μg/mL Zinc, 30 mg/mL Trehalose pH 4. 60 mL of pooled solution was added for dialysis.

Three dialysis solution exchanges were performed to remove greater than 99.9% of the excipients from the pooled solutions. Following dialysis, the resulting dialyzed solutions from each dialysis solution were pooled and concentration tested. Because the dialysis solutions were similar in tonicity to the tonicity of the commercially supplied insulin glargine, there was minimal change in concentration resulting from the dialyses.

For formulations 1, 2, 3, 4, 12, 13 and 14, the final dialysis buffer was identical to the previous dialysis solution, except that m-cresol was included at a concentration of 2.7 mg/mL. The final dialysis solutions for all formulations were as follows:
Formulations 1, 2, 3, 12, 13: 30 μg/mL Zinc, 20 mg/mL Glycerol, pH 4. 90 mL insulin glargine added for dialysis. Approximately 64 mL of LANTUS was added to the final dialysis solution.
Formulation 4: 30 μg/mL Zinc, 20 mg/mL Sorbitol, pH 4. 30 mL Lantus added for dialysis. Approximately 16 mL of Lantus was added to the final dialysis solution.
Formulations 5, 6: 30 μg/mL Zinc, 2.7 mg/mL m-cresol, 30 mg/mL trehalose, pH 4. Approximately 8 mL of Lantus was added to the final dialysis solution.
Formulation 7: 30 μg/mL Zinc, 2.7 mg/mL m-cresol, 30 mg/mL trehalose, 100 mM NaCl pH 4. Approximately 9 mL of Lantus was added to the final dialysis solution.
Formulation 8: 30 μg/mL Zinc, 2.7 mg/mL m-cresol, 30 mg/mL trehalose, 50 mM Arginine pH 4. Approximately 10 mL of Lantus was added to the final dialysis solution.
Formulation 9: 30 μg/mL Zinc, 2.7 mg/mL m-cresol, 30 mg/mL trehalose, 50 mM Proline pH 4. Approximately 19.5 mL of Lantus was added to the final dialysis solution.
Formulation 10: 30 μg/mL Zinc, 2.7 mg/mL m-cresol, 50 mM Arginine, 100 mM NaCl, pH 4. Approximately 10 mL of Lantus was added to the final dialysis solution.
Formulation 11: 30 μg/mL Zinc, 2.7 mg/mL m-cresol, 50 mM Proline, 100 mM NaCl, pH 4. Approximately 10 mL of Lantus was added to the final dialysis solution.

For surfactant containing formulations, surfactant stock solutions were prepared at a concentration of 1% w/v. The stock solutions were added to the dialyzed solutions for formulations 2-5 and 12-14 to obtain appropriate surfactant concentrations in the final formulation solution.

The final formulation solutions were sterile filtered and filled aseptically into 2 mL vials at a volume of about 400 μL/vial. Since polysorbate 20 was included in the commercially available vial formulation and not the cartridge formulation and the major difference between the vial and cartridge configuration is the container headspace. The greater headspace volume in the vial compared to the headspace volume in the cartridge may contribute to the aggregation of the insulin glargine in the vial upon agitation. The vial configuration used in these studies was intended to maximize the propensity for agitation-induced damage to the protein. All vials were labeled with the formulation number and the date of preparation.

Vials were placed on station under the following stress conditions for the listed time periods:

4° C. (real time)
No agitation, Time=0, 1, 2, and 4 week

25° C. (accelerated)
No agitation, Time=0, 1, 2, and 4 week
With agitation, Time=1 week

40° C.
No agitation, Time=0, 1, 2, and 4 week
With agitation, Time=1 and 2 week

Degradation at 40° C. does not always follow the same pathway as at lower temperatures. While data was collected for this temperature, the data analysis was focused primarily on the 4° C. and 25° C. data unless no differences in stability were observed for those temperatures. Agitated samples were placed on a platform rotator with a speed set to approximately 60 rpm inside of a stability chamber at the specified temperature. The following seven analytical methods were utilized in the evaluation of the different formulations: (1) Visual inspection: Samples were inspected for color, clarity, and the presence of particulate matter at each timepoint for each study condition; (2) Size Exclusion Chromatography-High Performance Liquid Chomatography (SEC-HPLC); (3) Cation Exchange-HPLC (CEX-HPLC: (4) Reverse Phase-HPLC (RP-HPLC); (5) Dynamic Light Scattering (DLS): DLS measurements were performed on a Malvern (ZEN3600, s/n MAL1034304) light scattering detector; (6) OD₃₅₀: OD₃₅₀ measurements were performed on a Varian Cary 100 UV/VIS spectrophotometer; and (7) Brightwell (Micro-Flow Imaging): Micro-Flow Imaging (MFI) is a technique used to determine particle sizes and counts in a sample. Samples are pumped through a 100 micron, 1.6 mm uncoated SP3 Flow Cell and high speed photographic images of particles detected through a digital microscope are captured and recorded for each sample. The technique is able to detect particles in a size range of 1 micron to 100 microns in size. This assay is not performed in a particulate controlled environment; therefore, it should be noted that contaminants from the air/surrounding environment could affect results.

### Results

### Visual Appearance

Visual appearance analysis was performed on all conditions at each time point. The majority of the vial sample solutions were clear, colorless, and free from particulate-containing matter at study initiation. The exception to this was formulation 12, which showed a slight haze upon visual inspection. This formulation contained a relatively high concentration of Span 40 (200 μg/mL). It is important to note that excipient buffer in the absence of protein for this formulation also showed haziness, suggesting that the Span may have limited solubility under these conditions.

No major changes in visual appearance were observed over the course of the study for any of the non-agitated vial samples for any of the temperatures studied (See Tables 3, 4, and 5). This was also true for the agitated vial samples stored at 25° C. For vial samples agitated at 40° C., a number of vial samples showed the appearance of particulates following two weeks agitation (See Table 6). Typically, the particles that were observed were white in color and fibril-like in shape.

TABLE 3
Appearance Results for Samples stored at 2-8° C., no agitation

| Formulation | | | | |
|---|---|---|---|---|
| Number | T = 0 | T = 1 week | T = 2 weeks | T = 4 weeks |
| 1 | CCF | CCF | CCF | CCF |
| 2 | CCF | CCF | CCF | CCF |
| 3 | CCF | CCF | CCF | CCF |
| 4 | CCF | CCF | CCF | CCF |
| 5 | CCF | CCF | CCF | CCF |
| 6 | CCF | CCF | CCF | CCF |
| 7 | CCF | CCF | CCF | CCF |
| 8 | CCF | CCF | CCF | CCF |
| 9 | CCF | CCF | CCF | CCF |
| 10 | CCF | CCF | CCF | CCF |
| 11 | CCF | CCF | CCF | CCF |
| 12 | Particulates | Particulates | Particulates | Particulates |
| | detected | detected | detected | detected |
| 13 | CCF | CCF | CCF | CCF |
| 14 | CCF | CCF | CCF | CCF |

CCF—Clear, Colorless, and Free of Particulates

TABLE 4
Appearance Results for Samples stored at 25° C., no agitation

| Formulation | | | T = 2 | T = 4 |
|---|---|---|---|---|
| Number | T = 0 | T = 1 week | weeks | weeks |
| 1 | CCF | CCF | CCF | CCF |
| 2 | CCF | CCF | CCF | CCF |
| 3 | CCF | CCF | CCF | CCF |
| 4 | CCF | CCF | CCF | CCF |
| 5 | CCF | CCF | CCF | CCF |
| 6 | CCF | CCF | CCF | CCF |
| 7 | CCF | CCF | CCF | CCF |
| 8 | CCF | CCF | CCF | CCF |
| 9 | CCF | CCF | CCF | CCF |
| 10 | CCF | CCF | CCF | CCF |
| 11 | CCF | CCF | CCF | CCF |
| 12 | Particulates | Particulates | Particulates | Particulates |
| | detected | detected | detected | detected |
| 13 | CCF | CCF | CCF | CCF |
| 14 | CCF | CCF | CCF | CCF |

CCF—Clear, Colorless, and Free of Particulates

TABLE 5
Appearance Results for Samples stored at 40° C., no agitation

| Formulation | | | | |
|---|---|---|---|---|
| Number | T = 0 | T = 1 week | T = 2 weeks | T = 4 weeks |
| 1 | CCF | CCF | CCF | CCF |
| 2 | CCF | CCF | CCF | CCF |
| 3 | CCF | CCF | CCF | CCF |
| 4 | CCF | CCF | CCF | CCF |
| 5 | CCF | CCF | CCF | CCF |
| 6 | CCF | CCF | CCF | CCF |
| 7 | CCF | CCF | CCF | CCF |
| 8 | CCF | CCF | CCF | CCF |
| 9 | CCF | CCF | CCF | CCF |
| 10 | CCF | CCF | CCF | CCF |
| 11 | CCF | CCF | CCF | CCF |
| 12 | Particulates | Particulates | Particulates | Particulates |
| | detected | detected | detected | detected |
| 13 | CCF | CCF | CCF | CCF |
| 14 | CCF | CCF | CCF | CCF |

CCF—Clear, Colorless, and Free of Particulates

TABLE 6
Appearance Results for Agitated Samples following
storage at 25° C. and 40° C.

| Formulation | T = 1 week, | T = 1 week, | T = 2 week, |
|---|---|---|---|
| Number | 25° C. | 40° C. | 40° C. |
| 1 | CCF | CCF | Particulates |
| | | | detected |
| 2 | CCF | CCF | CCF |
| 3 | CCF | CCF | Particulates |
| | | | detected |
| 4 | CCF | CCF | Particulates |
| | | | detected |
| 5 | CCF | CCF | Particulates |
| | | | detected |
| 6 | CCF | CCF | Particulates |
| | | | detected |
| 7 | CCF | CCF | CCF |
| 8 | CCF | CCF | CCF |
| 9 | CCF | CCF | Particulates |
| | | | detected |
| 10 | CCF | CCF | CCF |
| 11 | CCF | CCF | Particulates |
| | | | detected |
| 12 | Particulates | Particulates | Particulates |
| | detected | detected | detected |
| 13 | CCF | CCF | CCF |
| 14 | CCF | CCF | Particulates |
| | | | detected |

CCF—Clear, Colorless, and Free of Particulates

### SEC-HPLC

Size Exclusion Chromatography (SEC) is typically utilized to look for the formation of soluble aggregates, both covalent and non-covalent. The SEC-HPLC method utilized in the insulin glargine preformulation studies has a high acetic acid and organic content in the mobile phase. As such, it is unlikely that the method is as sensitive to the detection of non-covalent aggregates. However, the technique provides a measure of dimeric species (presumably covalent), as well as a measure of the formation of insoluble aggregates (such as fibrils), detected as a loss in monomer over the course of the study.

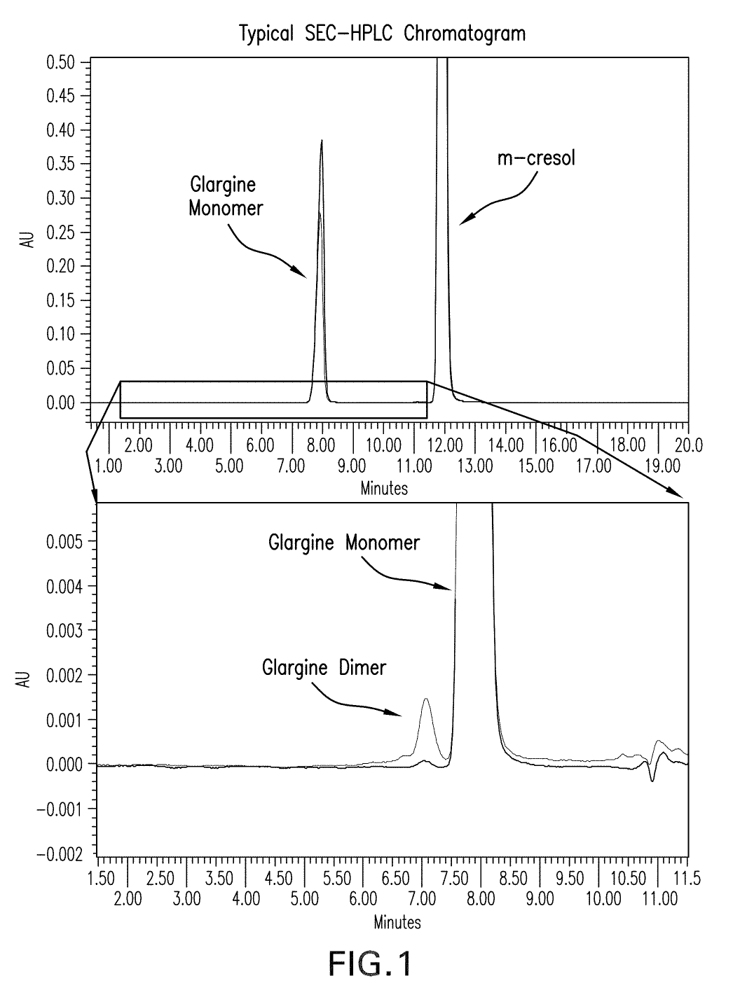

Typical chromatograms are shown in FIG. 1 below. In this figure, chromatograms of stressed (heat+agitation) vs. unstressed material are shown by the black and dotted-line traces respectively. The top chromatogram is a full scale image. A very large peak is observed in the assay at approximately 12 minutes. This has been identified as m-cresol through the analysis of excipient blank solutions. The other large peak, observed at approximately 8 minutes, is indicative of monomeric insulin glargine. The bottom chromatogram is a magnification of the baseline around the monomer peak. This shows the presence of a small peak at approximately seven minutes, which may represent covalent dimers.

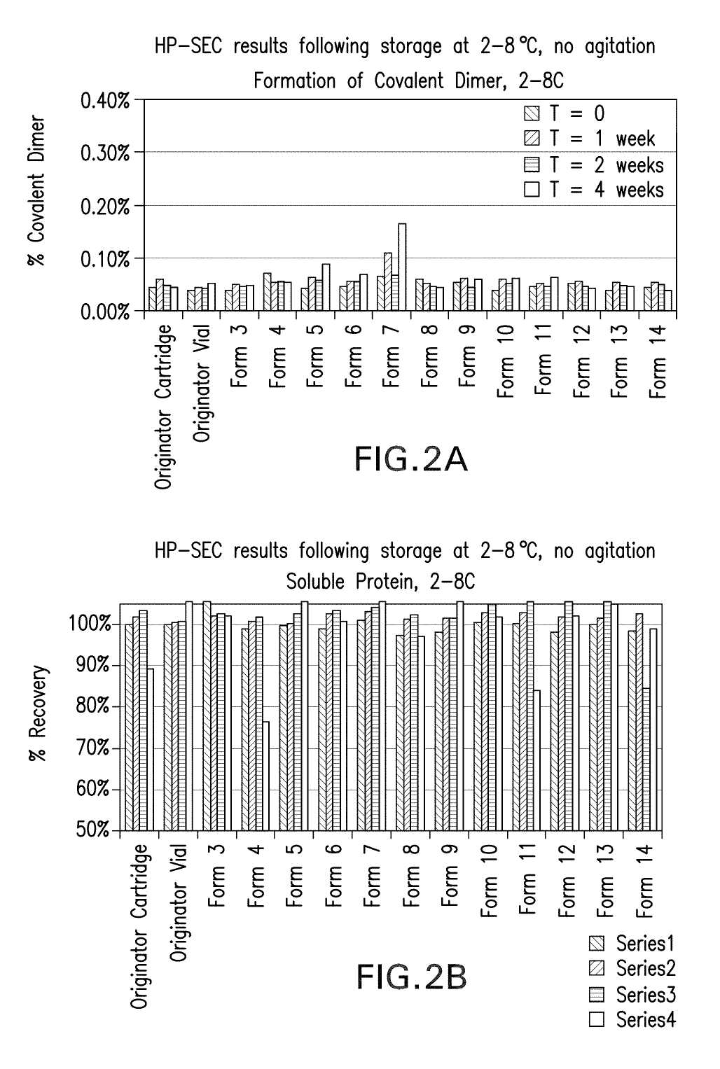

Analysis of the fourteen formulations showed essentially no detectable change in aggregate content for samples stored at 2-8° C. in the absence of agitation and the majority of the formulations showed essentially no increase in colvalent dimer over the course of the study and performed similarly to originator cartridge and vial formulations. Formulation 5 and formulation 7 seem to show a small increase in covalent dimer content after four weeks of storage, as illustrated in FIG. 2A. However, the qualification of this assay suggests a quantitation limit of 0.25%. FIG. 2B shows the recovery of soluble protein in each of the samples. While not a precise measurement (error typically in the range of +/−7%), the data indicate no major loss in soluble protein over the course of the study at this storage temperature, with the exception of Formulations 4 and 11.

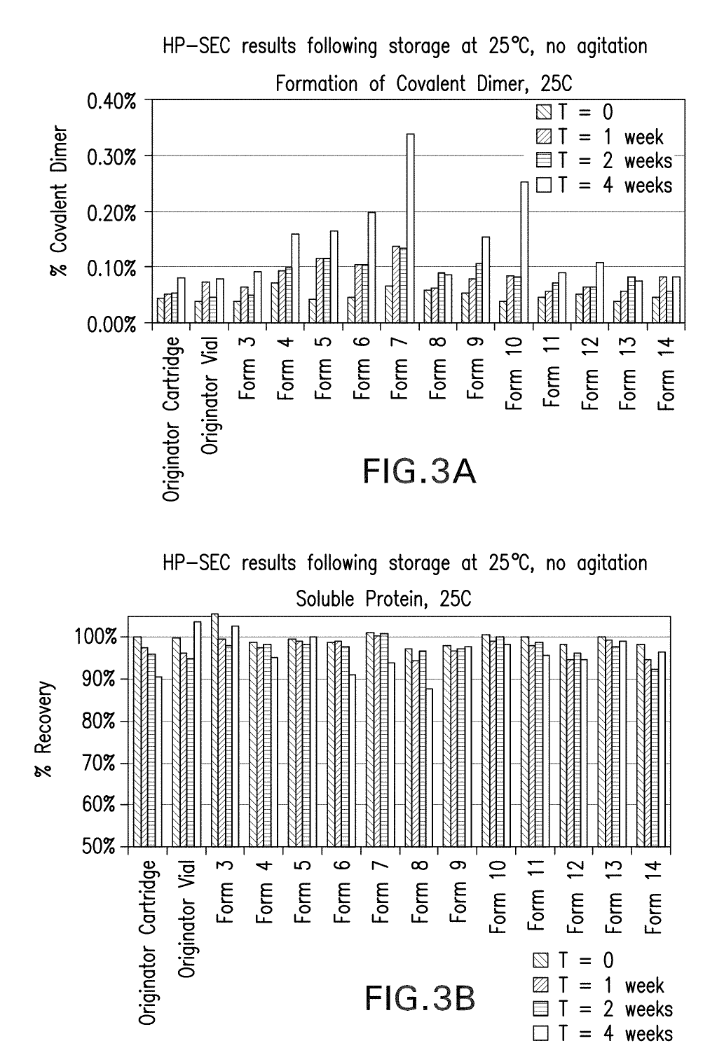

Storage at 25° C. in the absence of agitation showed an increase in covalent dimer formation in a number of the formulations following storage, as illustrated in FIG. 3B. Generally, the glycerol containing formulations showed better stability at this storage condition compared to the other formulations. FIG. 3B shows a loss in recovery for some of the formulations following storage, including the originator cartridge formulation and the formulations which contained trehalose in the absence of stabilizer.

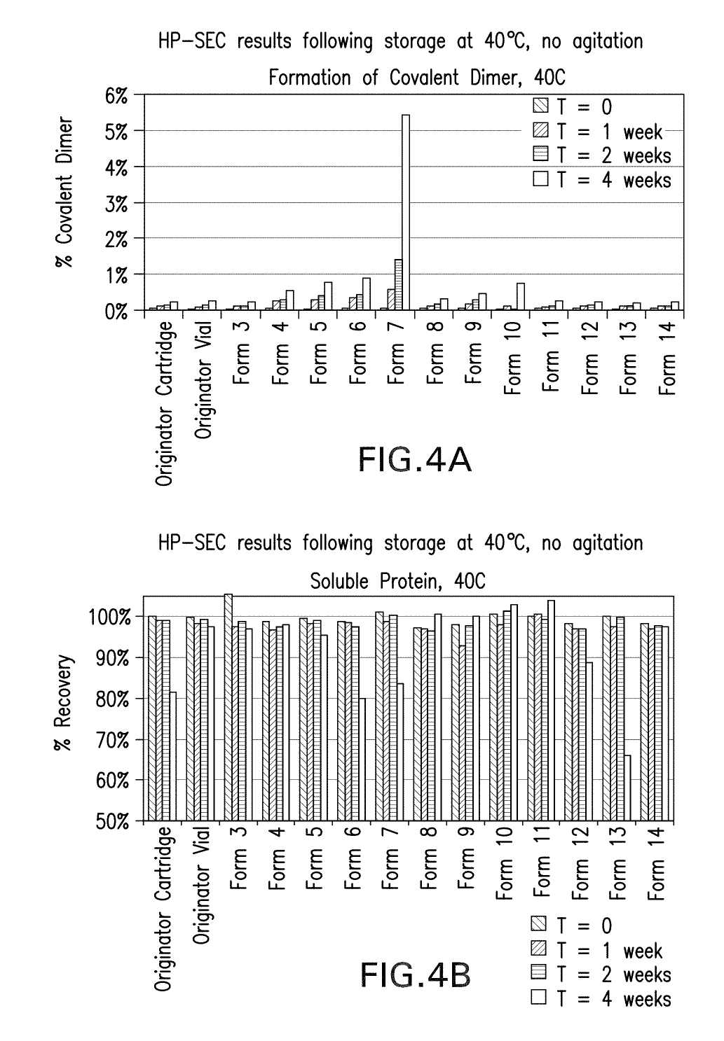

Storage at 40° C. in the absence of agitation showed also an increase in covalent dimer formation following storage for formulations that did not contain glycerol, as illustrated in FIG. 4A. FIG. 4B also showed similar results to the 25° C. data with losses in recovery following storage for formulations containing trehalose in the absence of surfactant. Loss in recovery was also observed for formulation 12, which contains glycerol as tonicity modifier and a high concentration of Span 40 for the surfactant.

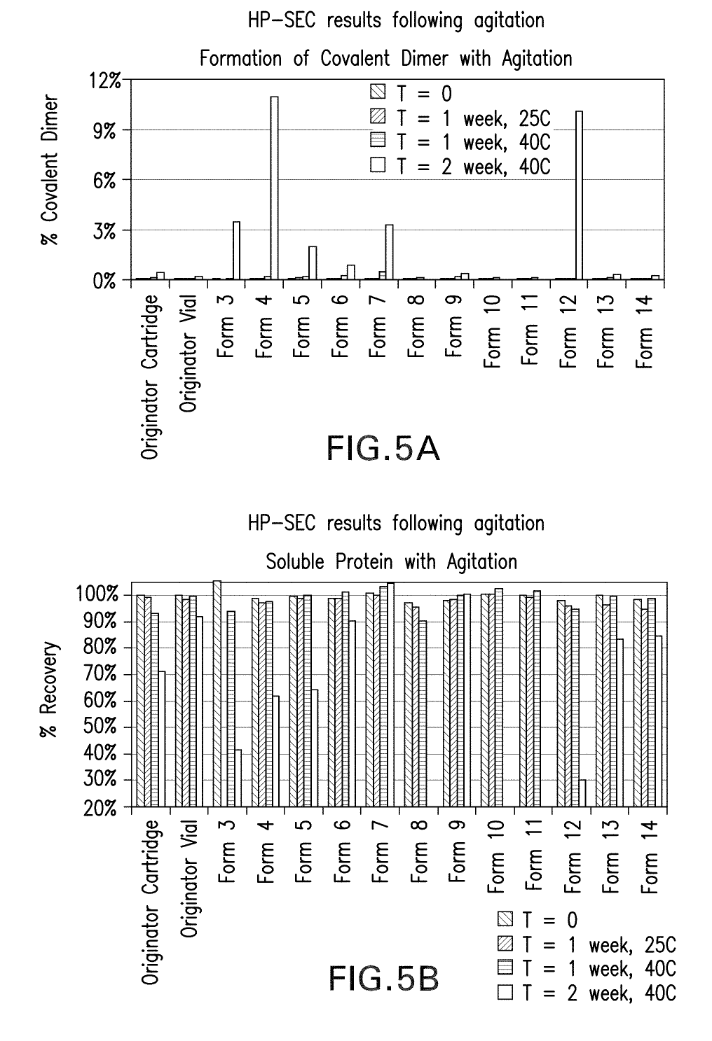

Agitation of the samples at 25° C. for one week did not result in much change in formation of insoluble aggregate or loss in protein recovery. Agitation of the samples at 40° C. for one week resulted in similar trends with respect to covalent aggregate formation as were seen in the absence of agitation, with glycerol containing formulations generally performing best. Following two weeks agitation at 40° C., significant increases in covalent dimer were observed, specifically in formulations that included trehalose as a tonicity modifier. The exception to that was the trehalose/proline formulation. Formulations that included Span 40 also showed increases in covalent dimer under this stress condition. Loss in soluble protein was observed following 2 weeks agitation at 40° C. in a number of samples. These results are shown in FIGS. 5A and 5B.

A few samples, specifically formulations 8, 10, and 11 (Trehalose/Arginine, NaCl/Arginine, and NaCl/Proline respectively) could not be analyzed by any of the HPLC assays following two weeks agitation at 40° C. due to gellation of the samples.

### RP-HPLC

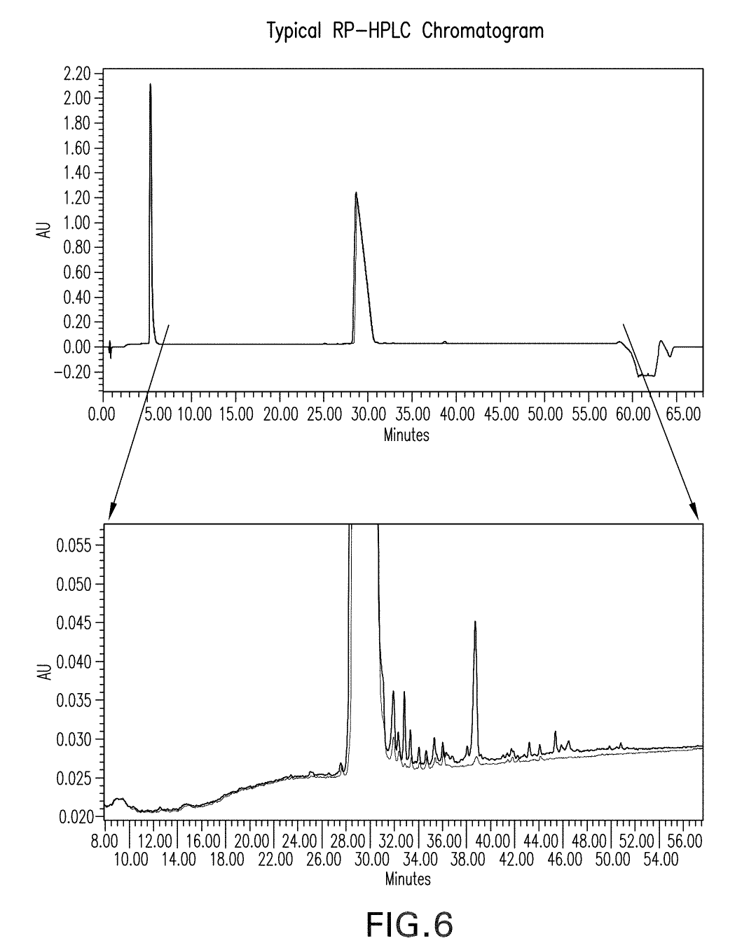

Reversed Phase HPLC is typically utilized to look impurity species that differ in hydrophobicity compared to the pure protein. Typical chromatograms are shown in FIG. 6 below. In this figure, chromatograms of stressed (heat+agitation) vs. unstressed material are shown by the dotted-line and black traces respectively. The top chromatogram is a full scale image. Excipients, including m-cresol, general elute very early (typically prior to seven minutes. The large peak, observed at approximately 30 minutes, is indicative of insulin glargine. The majority of impurity peaks are observed on the backside of the main insulin glargine peak, as shown in the bottom image in FIG. 6.

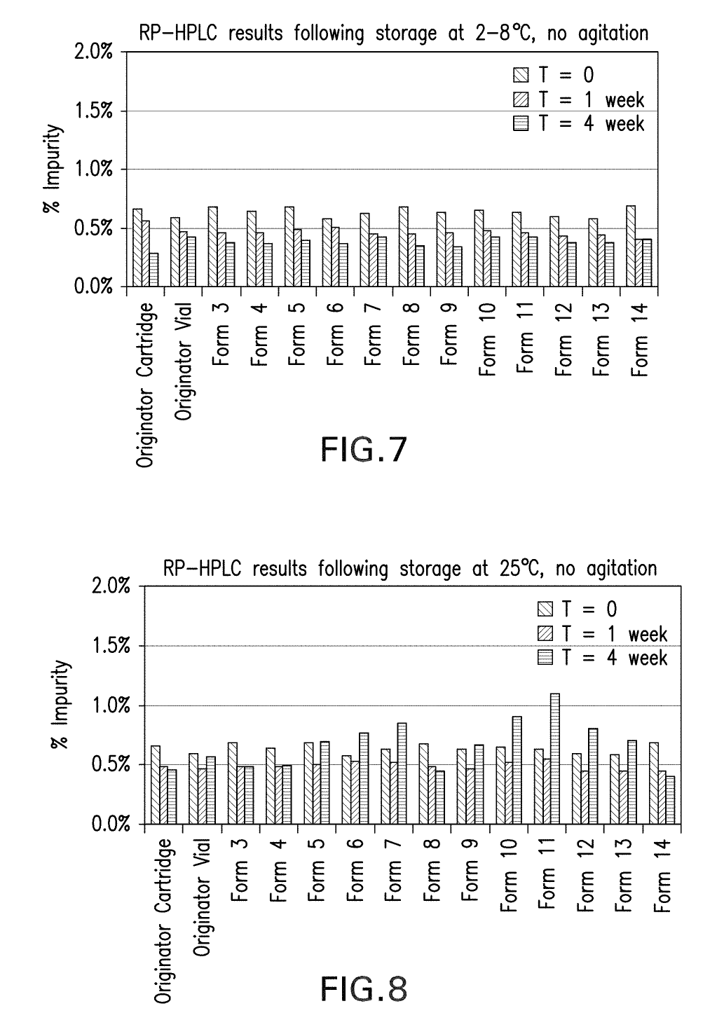

Analysis of the fourteen formulations showed essentially no change in impurities over the course of the study for samples stored at 2-8° C. in the absence of agitation, as illustrated in FIG. 7. The results were similar at 25° C. with the exception of the NaCl containing formulations (numbers 7, 10 and 11), which did show increases in impurity levels over the course of the study, as shown in FIG. 8.

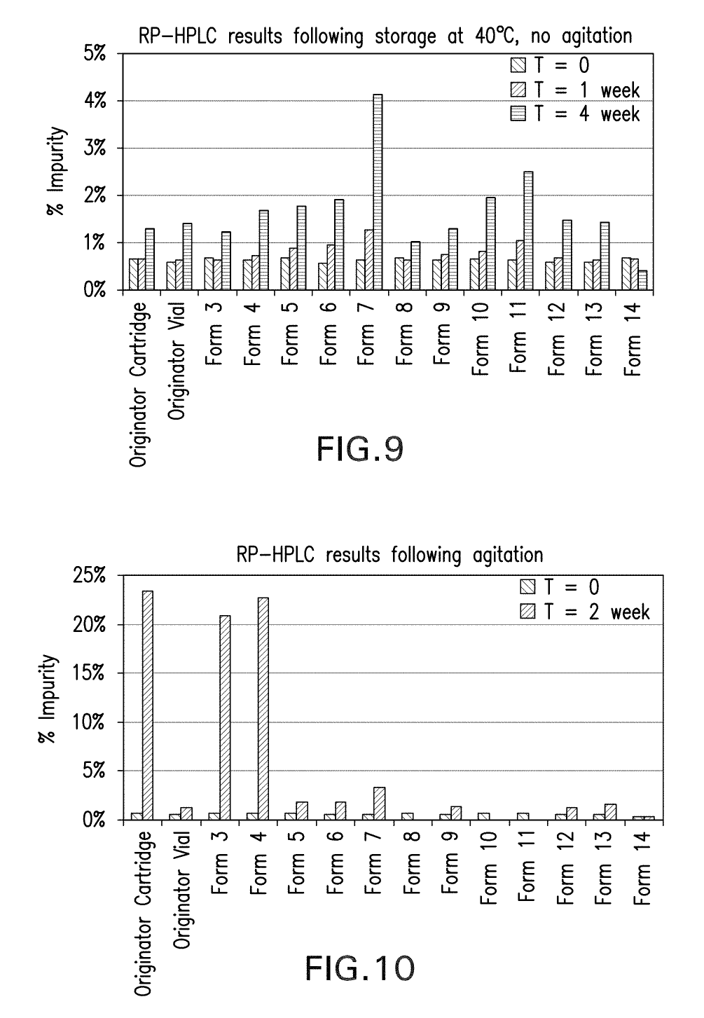

Increases in impurity levels were observed over the course of the study for samples stored at 40° C., both with and without agitation, as illustrated in FIG. 9 and FIG. 10. In the absence of agitation, samples that did not contain glycerol showed increases in impurities. This is similar to the trends observed by HP-SEC. Agitated samples showed similar levels of impurity in most samples to the non-agitated samples. However, a few formulations, including originator cartridge (which contains glycerol but no surfactant) and the Span 40 containing formulations, showed a dramatic increase in impurity by RP-HPLC over the course of the study.

### CEX-HPLC

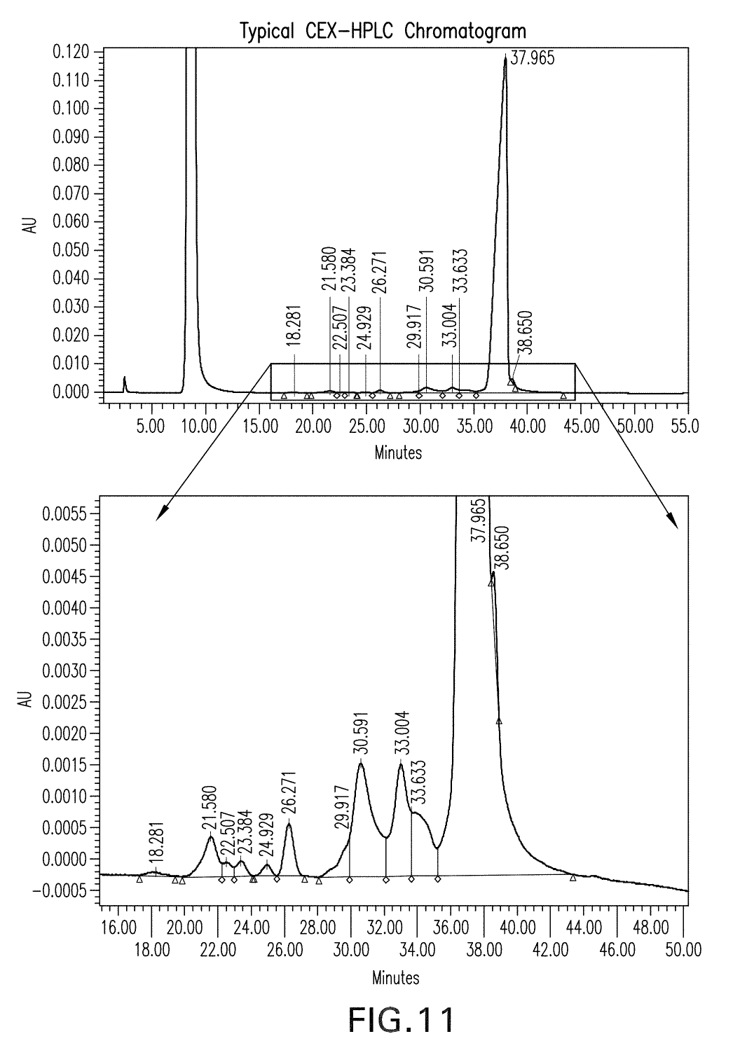

Cation Exchange HPLC is typically utilized to look impurity species that differ in charge compared to the pure protein. Typical chromatograms are shown in FIG. 11 below. The top chromatogram is a full scale image. Excipients, including m-cresol, generally elute prior to 11 minutes. The large peak, observed at approximately 38 minutes, is indicative of insulin glargine. The majority of impurity peaks are observed on the frontside of the main insulin glargine peak between 30 and 35 minutes, as shown in the bottom image in FIG. 11. The appearance of peaks prior to the main peak when analyzed by CEX-HPLC suggests degraded forms that are more acidic in nature. Backside peaks, which are more basic than the main peak, are difficult to quantitate due to their appearance as a backside shoulder to the main peak.

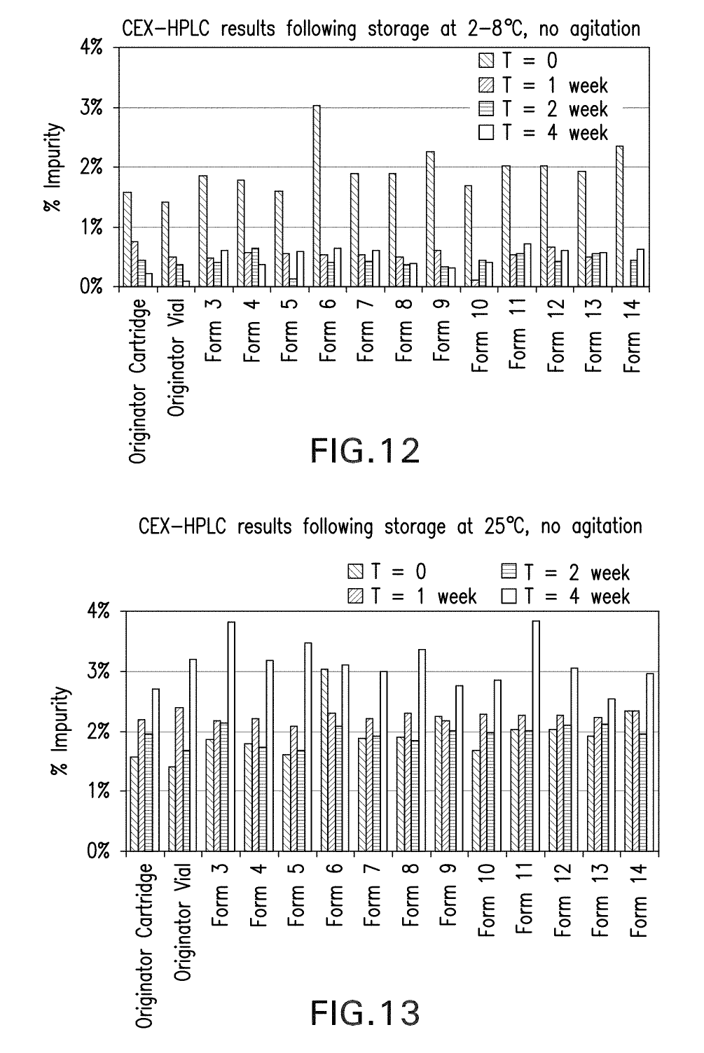

Analysis of the fourteen formulations stored at 2-8° C. in the absence of agitation, shown in FIG. 12, seems to suggest a decrease in aggregation over time. However, this is most likely indicative of the error of the method for relatively low levels of impurity. Qualification of this assay indicates an LOQ of approximately 1% impurity. The biggest issues in impurity quantitation are the peaks that are not well resolved from the main peak. The backside peak, although shown in this example as a tangent skim integration, was generally integrated with a drop straight to the baseline when it was observed, so the reported results likely err on the high side in cases where this backside shoulder was integrated.

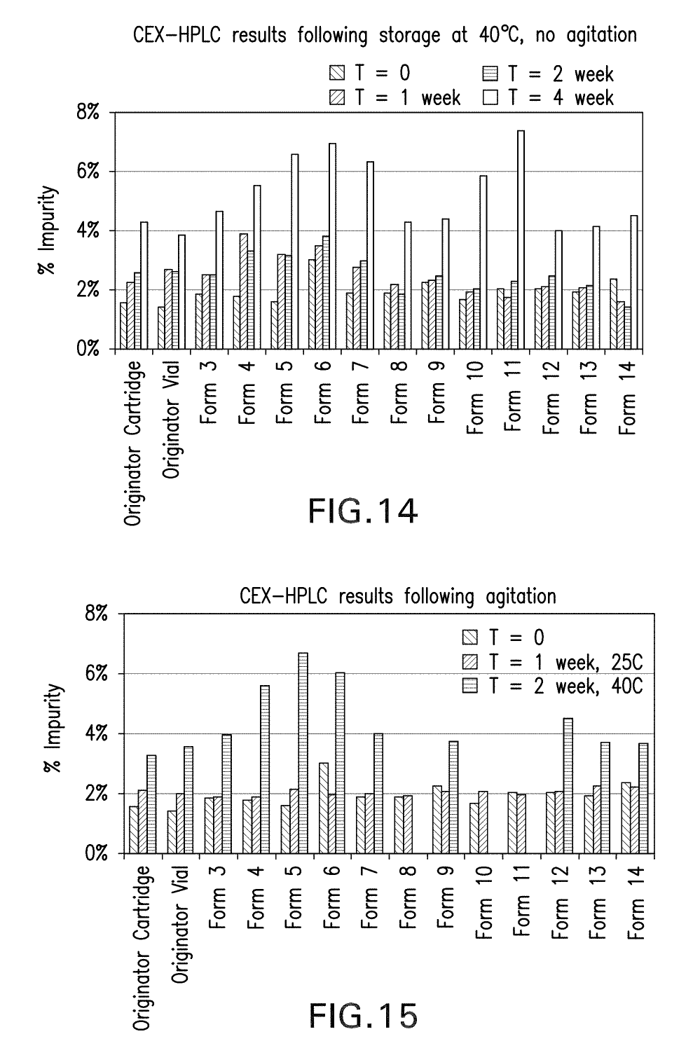

The results for samples stored at 25° C. and 40° C. in the absence of agitation show increases in charge variants for all formulations over the course of the study (FIG. 13 and FIG. 14). The trends observed by CEX-HPLC are very similar to those observed by RP-HPLC, with respect to glycerol containing formulations generally performing better than those with alternate tonicity modifiers. Agitation does appear to increase the rate of formation of the charge variant species, as shown in FIG. 15.

### OD350

An analysis of each formulation was performed at all time points of the study looking at absorbance of the samples following UV/Vis analysis at a wavelength of 350 nm. Increases in absorbance at 350 nm could be indicative of aggregate formation, manifested as an increase in light scattering.

The only formulation that showed an OD350 reading above the noise level was formulation 12. This is the formulation containing the high concentration of Span 40, and which was observed to have particulate matter present upon visual inspection in both protein containing samples and excipient blanks. The particulates observed during visual inspection would be expected to result in an increase in light scattering and therefore absorbance at 350 nm.

### Brightwell Microflow Imaging

Microflow Imaging was performed on a Brightwell (DPA-4200, s/n 000701). This instrument has been demonstrated in the literature to be capable of detecting subvisible particles, and appears to have a greater sensitivity than more traditional techniques such as HIAC. An additional advantage to this technique is the sample volume requirements—Brightwell analysis can be performed on solution volumes as low as 750 μL. In addition to counting particles, the Brightwell instrument provides images of the observed particles. This allows, in many cases, a distinction between protein aggregates and/or fibrils and other types of particles (fibers, hairs, air bubbles, silicone oil droplets, etc) due to differences in color, shape, and intensity.

The Brightwell method is very sensitive to sample handling and contamination. In this study, volumes were very limited due to the large number of formulations and conditions evaluated. A single vial was used for all of the asssays discussed above, and the remainder of the solution in that vial was analyzed by Brightwell. So, there is a strong potential for sample contamination with particulate matter from the air and surfaces (i.e., pipet tips) that may have come into contact with the solutions.

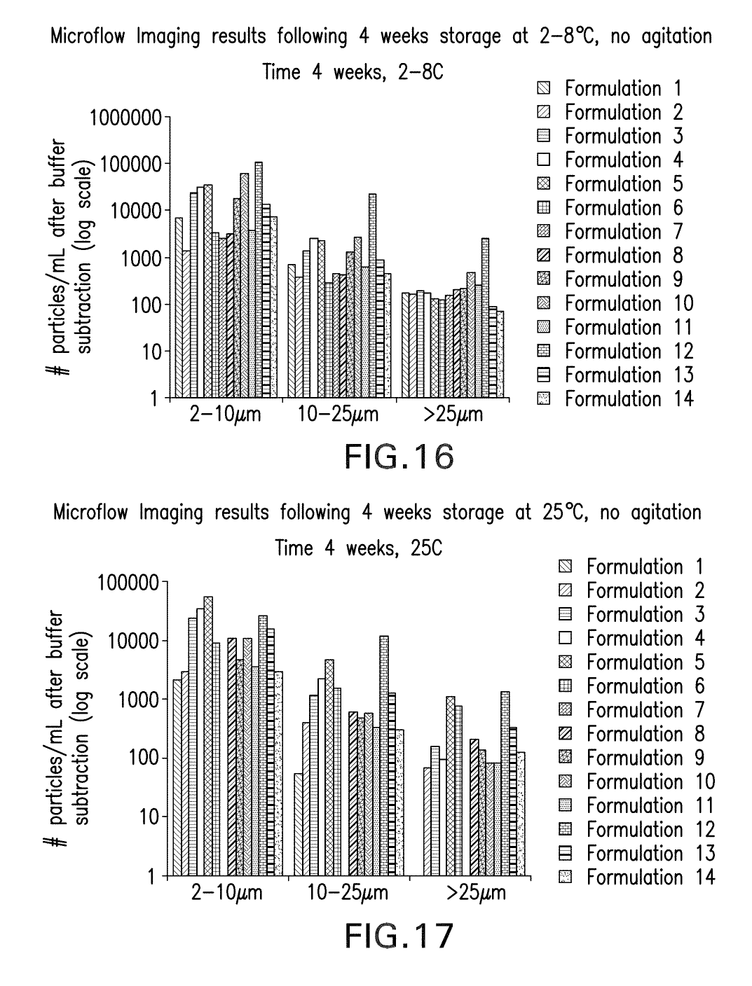

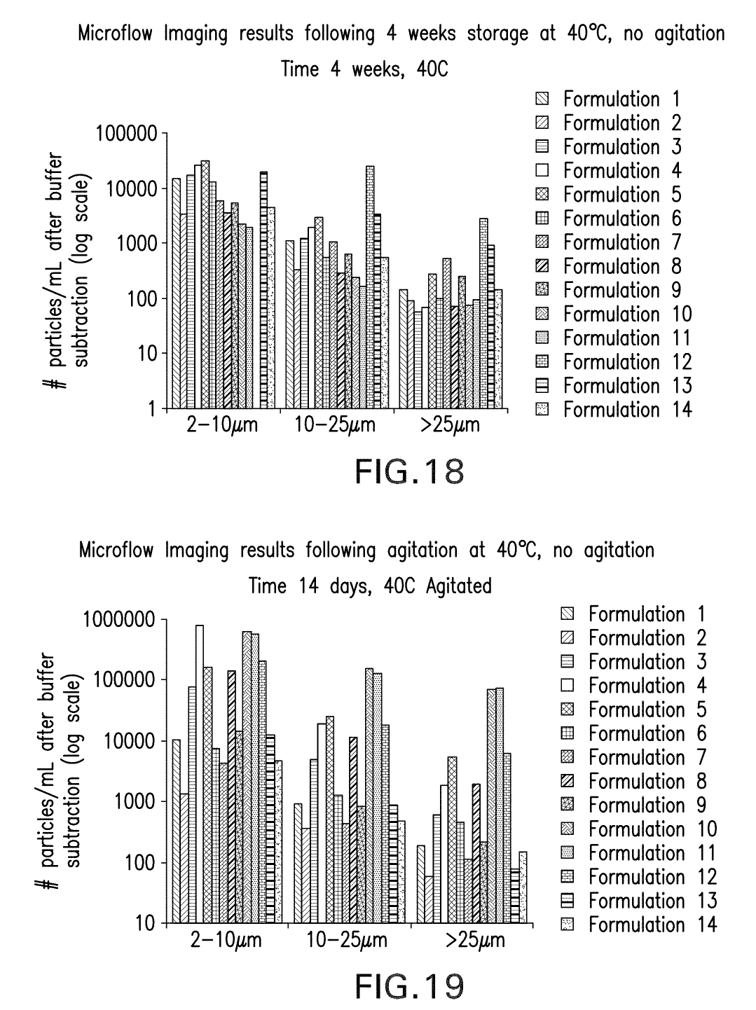

Results from the Brightwell analysis did not show any major trends over the course of the study for samples that were not agitated. Representative plots of the data for samples held at 2-8° C., 25° C., and 40° C. in the absence of agitation are presented in FIG. 16, FIG. 17, and FIG. 18. The only formulation that was really notable in this part of the study was formulation 12, which showed much higher particle counts in the greater than 10 μm range than the other samples. This is not surprising since this is the formulation containing the high Span 40 concentration in which particles were observed upon visual inspection.

Several of the formulations showed increased particle counts in the agitation studies, especially at 40° C., as shown in FIG. 19. Particle counts for some formulations were an order of magnitude higher than for non-agitated samples. The samples that were of greatest concern were formulations 8, 10 and 11. This is not surprising since these formulations formed gels over the course of the study and had to be “resuspended” for analysis. Also showing increased particle count upon agitation were the Span 40-containing formulations (formulation 3, 4, 5 and 12).

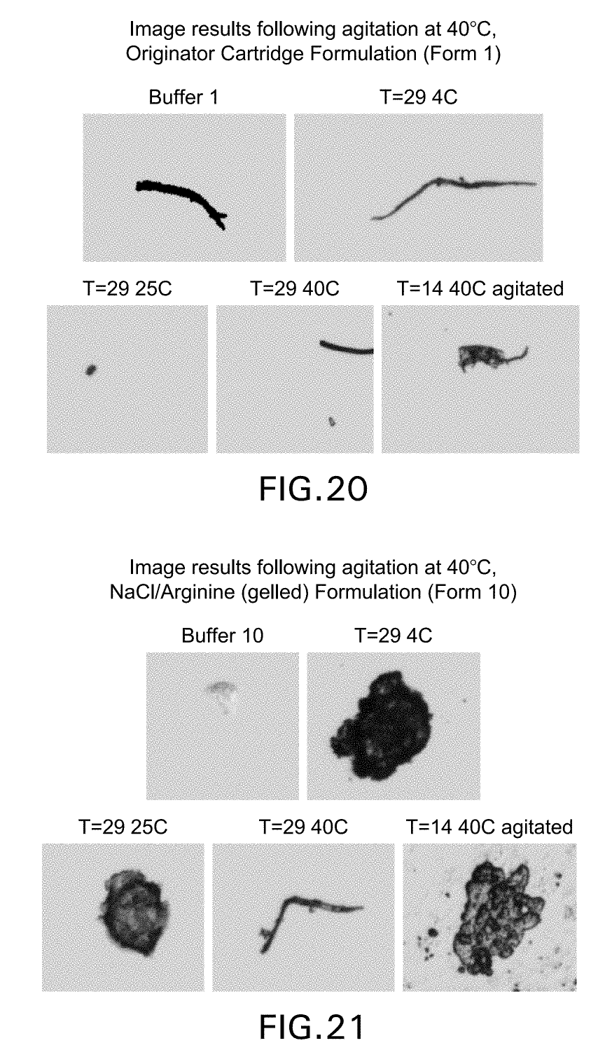

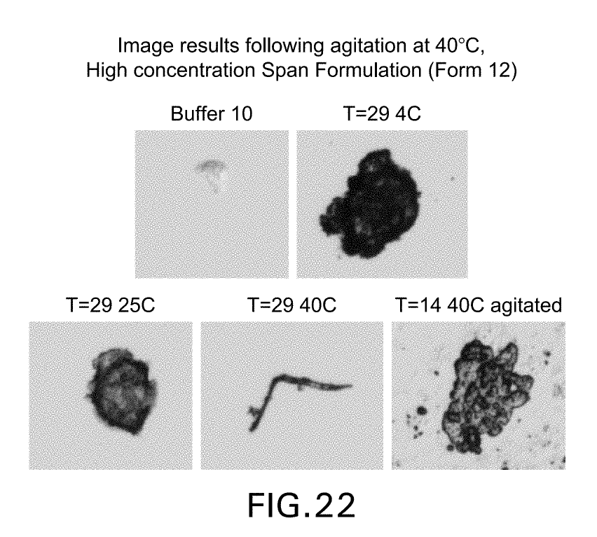

A number of different types of particles were observed in the images produced by the Brightwell instrument, including fibers, fibrils, agglomerates, and others that can not be uniquely identified. Images for some representative formulations (excipient buffers compared to stability samples) are shown in FIG. 20 to FIG. 22.

### Dynamic Light Scattering (DLS)

DLS measurements were performed on a Malvern (ZEN3600, s/n MAL1034304) light scattering detector. Like the OD350 measurements, analysis of light scattering in a sample may be indicative of aggregate formation. No trends were observed by DLS over the course of the preformulation study.

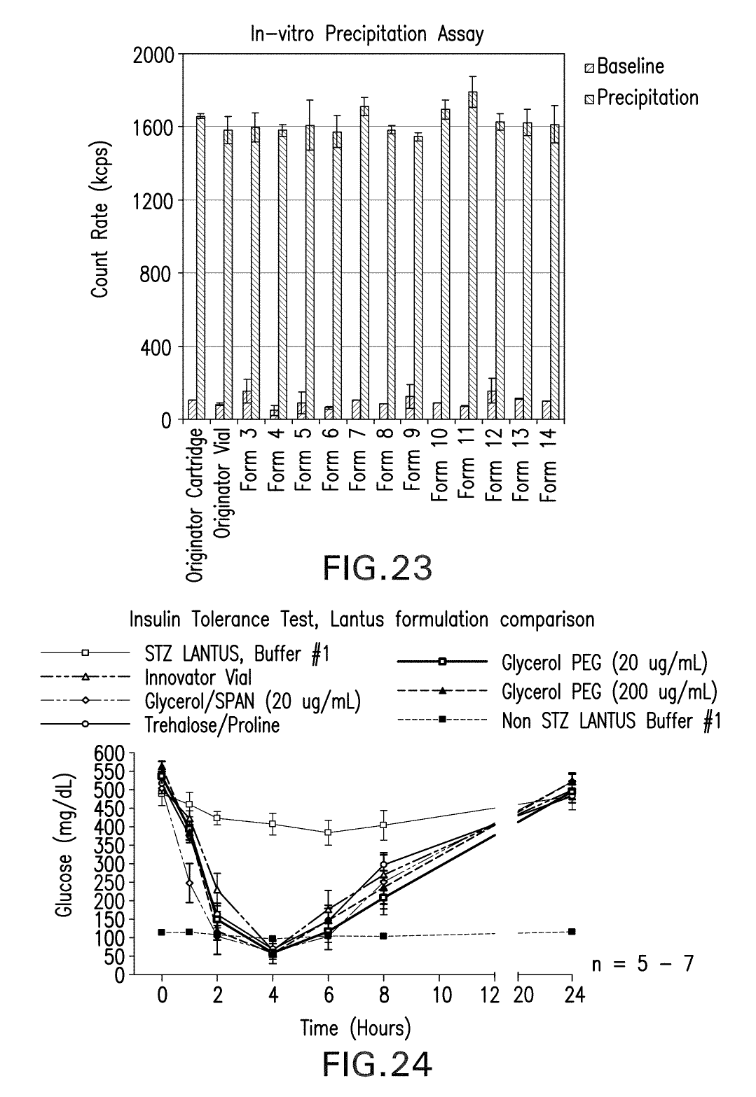

In addition to the use of DLS to evaluate the formulated samples following storage, DLS was also utilized to evaluate depot formation for the formulations. A successful formulation will not only provide adequate shelf life for the vial but the insulin glargine will also form a depot upon injection into a patient. While a direct evaluation of depot formation in an animal model was not available at the time of this study. However, an in-vitro assay was developed to evaluate depot formation. Essentially, an aliquot of the formulated drug product was diluted into pH 7.4 phosphate buffer and the sample analyzed for changes in light scattering. Based on the results of the in-vitro assay, all formulations included in this study are capable of depot formation in the model system, as illustrated in FIG. 23.

### Example 2

The stability of insulin glargine in 30 mg/mL Trehalose with 50 mM Proline (30Tre/50Pro) or 200 μg/mL PEG-400 (20GLY/200PEG) was determined. The formulations comprise 3.65 mg/mL Insulin Glargine in 30 μg/mL Zn, 2.7 mg/mL m-cresol, 20 mg/mL (85%) Glycerol, pH 4 and either 30 mg/mL Trehalose and 50 mM Proline or 200 μg/mL PEG-400. Storage conditions were at the recommended storage for insulin glargine (2-8° C.) as well as under accelerated conditions (30° C. and 40° C.). The results are shown in Tables 7-12.

TABLE 7
Stability Data for insulin glargine preparation with the following formulation 20GLY/200PEG Storage at 2-8° C.

| Test | | Interval (Months) and Target Dates - Storage at 2-8° C. | | | | | | | |
|---|---|---|---|---|---|---|---|---|---|
| (Method) | Specification | Initial result | 1 month | 2 month | 3 month | 6 month | 9 month | 12 month | 18 month |
| Protein | *1-10 mg/mL | 3.70 | 3.95 | 3.72 | 3.71 | 3.74 | 3.65 | 3.65 | 3.61 |
| Concentration | | | | | | | | | |
| by SEC-HPLC | | | | | | | | | |
| Aggregates by | Purity ≧98.0% | 100.00% | 99.96% | 99.97% | 99.95% | 99.96% | 99.97% | 99.92% | 99.9%** |
| HPSEC | Impurity | <0.08 | <0.08 | <0.08 | <0.08 | <0.08 | <0.08 | <0.25 | <0.25 |
| | <2.0% | | | | | | | | |
| | LOD = 0.08 | | | | | | | | |
| Purity by | Report result | 99.16% | 99.30% | 99.17% | 99.00% | 99.46% | 98.85% | 99.5%** | 99.6%** |
| RP-UPLC | | | | | | | | | |
| Main Charge | ≧97.0% | 99.90% | 99.83% | 99.89% | 99.34% | 99.05% | 99.51% | 100.00% | 100.0%** |
| Variants by | intact | | | | | | | | |
| CEX-HPLC | | | | | | | | | |

*Although concentration specifications listed as a whole number, additional significant figures are reported for stability trending.
**Results reported to one decimal place

TABLE 8
Stability Data for insulin glargine preparation with the following formulation 30Tre/50Pro Storage at 2-8° C.

| Test | | Interval (Months) and Target Dates - Storage at 2-8° C. | | | | | | | |
|---|---|---|---|---|---|---|---|---|---|
| (Method) | Specification | Initial result | 1 month | 2 month | 3 month | 6 month | 9 month | 12 month | 18 month |
| Protein | *1-10 mg/mL | 3.65 | 4.05 | 3.72 | 3.72 | 3.67 | 3.65 | 3.63 | 3.58 |
| Concentration | | | | | | | | | |
| by SEC-HPLC | | | | | | | | | |
| Aggregates by | Purity ≧98.0% | 100.00% | 99.96% | 99.96% | 99.96% | 99.96% | 99.93% | 99.88% | 99.9%** |
| HPSEC | Impurity | <0.08 | <0.08 | <0.08 | <0.08 | <0.08 | <0.08 | <0.25 | <0.25 |
| | <2.0% | | | | | | | | |
| | LOD = 0.08 | | | | | | | | |
| Purity by | Report result | 99.14% | 99.33% | 99.05% | 98.87% | 98.93% | 98.90% | 99.5%** | 99.5%** |
| RP-UPLC | | | | | | | | | |
| Main Charge | ≧97.0% | 99.90% | 99.80% | 99.84% | 99.51% | 99.44% | 99.48% | 100.00% | 100.0%** |
| Variants by | intact | | | | | | | | |
| CEX-HPLC | | | | | | | | | |

*Although concentration specifications listed as a whole number, additional significant figures are reported for stability trending.
**Results reported to one decimal place

TABLE 9
Stability Data for MK-1293 with the following formulation 20GLY/200PEG, Storage at 30° C.

| Test | | Interval (Months) and Target Dates - Storage at 30° C. | | | | | | |
|---|---|---|---|---|---|---|---|---|
| (Method) | Specification | Initial result | 0.5 month | 1 month | 2 month | 3 month | 6 month | 9 month |
| Protein | *1-10 mg/mL | 3.70 | 3.66 | 3.96 | 3.62 | 3.72 | 3.71 | 3.72 |
| Concentration | | | | | | | | |
| by SEC-HPLC | | | | | | | | |
| Aggregates by | Purity ≧98.0% | 100.00% | 99.93% | 99.84% | 99.75% | 99.56% | 99.22% | 97.74% |
| HPSEC | Impurity | <0.08 | <0.08 | 0.16% | 0.25% | 0.44% | 0.78% | 2.26% |
| | <2.0% | | | | | | | |
| | LOD = 0.08 | | | | | | | |
| Purity by | Report result | 99.16% | 99.34% | 98.99% | 98.40% | 97.91% | 97.06% | 93.25% |
| RP-UPLC | | | | | | | | |
| Main Charge | ≧97.0% | 99.90% | 99.39% | 96.85% | 96.38% | 95.28% | 91.39% | 87.44% |
| Variants by | intact | | | | | | | |
| CEX-HPLC | | | | | | | | |

*Although concentration specifications listed as a whole number, additional significant figures are reported for stability trending.
**Results reported to one decimal place

TABLE 10
Stability Data for MK-1293 with the following formulation 30Tre/50Pro, Storage at 30° C.

| Test | | Interval (Months) and Target Dates - Storage at 30° C. | | | | | | |
|---|---|---|---|---|---|---|---|---|
| (Method) | Specification | Initial result | 0.5 month | 1 month | 2 month | 3 month | 6 month | 9 month |
| Protein | *1-10 mg/mL | 3.65 | 3.64 | 3.96 | 3.67 | 3.72 | 3.72 | 3.77 |
| Concentration | | | | | | | | |
| by SEC-HPLC | | | | | | | | |
| Aggregates by | Purity ≧98.0% | 100.00% | 99.92% | 99.87% | 99.64% | 99.32% | 98.41% | 96.08% |
| HPSEC | Impurity | <0.08 | 0.08% | 0.13% | 0.36% | 0.68% | 1.59% | 3.94% |
| | <2.0% | | | | | | | |
| | LOD = 0.08 | | | | | | | |
| Purity by | Report result | 99.14% | 99.35% | 99.09% | 98.48% | 97.96% | 95.81% | 92.58% |
| RP-UPLC | | | | | | | | |
| Main Charge | ≧97.0% | 99.90% | 99.25% | 96.80% | 96.75% | 95.46% | 92.52% | 88.82% |
| Variants by | intact | | | | | | | |
| CEX-HPLC | | | | | | | | |

*Although concentration specifications listed as a whole number, additional significant figures are reported for stability trending.
**Results reported to one decimal place

TABLE 11
Stability Data for MK-1293 with the following formulation
20GLY/200PEG, Storage at 40° C.

| | | Interval (Months) and Target Dates - | | | |
|---|---|---|---|---|---|
| | | Storage at 40° C. | | | |
| Test | | Initial | | | |
| (Method) | Specification | result | 1 month | 3 month | 6 month |
| Protein | *1-10 mg/mL | 3.70 | 4.13 | 3.92 | 4.05 |
| Concentration | | | | | |
| by SEC-HPLC | | | | | |
| Aggregates by | Purity ≧98.0% | 100.00% | 99.21% | 95.91% | 90.13% |
| HPSEC | Impurity | <0.08 | 0.79% | 4.09% | 9.87% |
| | <2.0% | | | | |
| | LOD = 0.08 | | | | |
| Purity by | Report result | 99.16% | 97.05% | 92.77% | 85.41% |
| RP-UPLC | | | | | |
| Main Charge | ≧97.0% | 99.90% | 91.71% | 80.48% | 65.06% |
| Variants by | intact | | | | |
| CEX-HPLC | | | | | |

*Although concentration specifications listed as a whole number, additional significant figures are reported for stability trending.
**Results reported to one decimal place

TABLE 12
Stability Data for MK-1293 with the following formulation
30Tre/50Pro, Storage at 40° C.

| | | Interval (Months) and Target Dates - | | | |
|---|---|---|---|---|---|
| | | Storage at 40° C. | | | |
| Test | | Initial | | | |
| (Method) | Specification | result | 1 month | 3 month | 6 month |
| Protein | *1-10 mg/mL | 3.65 | 4.06 | 3.95 | 4.02 |
| Concentration | | | | | |
| by SEC-HPLC | | | | | |
| Aggregates by | Purity ≧98.0% | 100.00% | 99.26% | 95.21% | 88.99% |
| HPSEC | Impurity | <0.08 | 0.74% | 4.79% | 11.01% |
| | <2.0% | | | | |
| | LOD = 0.08 | | | | |
| Purity by | Report result | 99.14% | 97.59% | 93.88% | 86.63% |
| RP-UPLC | | | | | |
| Main Charge | ≧97.0% | 99.90% | 92.62% | 82.86% | 70.87% |
| Variants by | intact | | | | |
| CEX-HPLC | | | | | |

*Although concentration specifications listed as a whole number, additional significant figures are reported for stability trending.
**Results reported to one decimal place

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An aqueous pharmaceutical formulation comprising
Gly(A21), Arg(B31), Arg(B32)-human insulin and
polyethylene glycol MW 400 (PEG 400); and
at least one preservative, wherein the aqueous pharmaceutical formulation has a pH in the acidic range from 1 to 6.8.

2. The aqueous pharmaceutical formulation as claimed in claim 1, wherein the at least one preservative is m-cresol.

3. The aqueous pharmaceutical formulation as claimed in claim 1, further including zinc.

4. The aqueous pharmaceutical formulation as claimed in claim 1, wherein the pharmaceutical formulation has a pH in the acidic range from 3.5 to 6.8.

5. The aqueous pharmaceutical formulation as claimed in claimed 4, wherein the pharmaceutical formulation has a pH in the acidic range from 3.5 to 4.5.

6. The aqueous pharmaceutical formulation as claimed in claim 1, wherein the Gly(A21), Arg(B31), Arg(B32)-human insulin is present in a concentration of about 100 to 1000 U/mL.

7. The aqueous pharmaceutical formulation as claimed in claim 6, wherein the Gly(A21), Arg(B31), Arg(B32)-human insulin is present in a concentration of about 100 or 300 U/mL.

8. The aqueous pharmaceutical formulation as claimed in claim 1, wherein the PEG MW 400 is present in a concentration of about 5-400 μg/mL.

9. The aqueous pharmaceutical formulation as claimed in claim 9, wherein the PEG MW 400 is present in a concentration of about 20 μg/mL.

10. An aqueous pharmaceutical formulation comprising
Gly(A21), Arg(B31), Arg(B32)-human insulin and
trehalose and proline; and
at least one preservative, wherein the aqueous pharmaceutical formulation has a pH in the acidic range from 1 to 6.8.

11. The aqueous pharmaceutical formulation as claimed in claim 10, wherein the at least one preservative is m-cresol.

12. The aqueous pharmaceutical formulation as claimed in claim 10, further including zinc.

13. The aqueous pharmaceutical formulation as claimed in claim 10, wherein the pharmaceutical formulation has a pH in the acidic range from 3.5 to 6.8.

14. The aqueous pharmaceutical formulation as claimed in claimed 13, wherein the pharmaceutical formulation has a pH in the acidic range from 3.5 to 4.5.

15. The aqueous pharmaceutical formulation as claimed in claim 10, wherein the Gly(A21), Arg(B31), Arg(B32)-human insulin is present in a concentration of about 100 to 1000 U/mL.

16. The aqueous pharmaceutical formulation as claimed in claim 15, wherein the Gly(A21), Arg(B31), Arg(B32)-human insulin is present in a concentration of about 100 or 300 U/mL.

17. The aqueous pharmaceutical formulation as claimed in claim 10, wherein the trehalose is present in a concentration of about 30 mg/mL.

18. The aqueous pharmaceutical formulation as claimed in claim 14, wherein the proline is present in a concentration of about 100-400 mM.

19. An aqueous pharmaceutical formulation comprising
An insulin derivative of the Formula 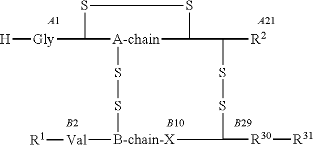
in which R¹ at position B1 denotes H or H-Phe; R² at position A21 denotes a genetically encodable L-amino acid selected from the group consisting of Gly, Ala, Val, Leu, Ile, Pro, Phe, Trp, Met, Ser, Thr, Cys, Tyr, Asp, and Glu; R³⁰ represents the residue of a neutral genetically encodable L-amino acid selected from the group consisting of Ala, Thr, and Ser; R³¹ represents 1, 2, or 3 neutral or basic α-amino acids, wherein at least one of the α-amino acids is selected from the group consisting of Arg, Lys, Hyl, Orn, Cit, and His; X represents His at position B10; and the sequences A1 to A20 and B1 to B29 in the Formula correspond to a mammalian insulin, excluding those insulin derivatives in which simultaneously: R¹ at position B1 denotes Phe; and R³¹ is one α-amino acid having a terminal carboxyl group; and optionally, the amino acid(s) at position A21 and/or the C-terminal amino acid of R³¹ is(are) amidated at the carboxy terminus; either (i) polyethylene glycol MW 400 or (ii) trehalose and proline; and
at least one preservative, wherein the aqueous pharmaceutical formulation has a pH in the acidic range from 1 to 6.8.

20. The aqueous pharmaceutical formulation of claim 19, wherein (i) the PEG MW 400 is present in a concentration of about 20 μg/mL or (ii) the trehalose is at a concentration of 30 mg/mL and the proline is at a concentration of 50 mM.
